(19) United States
(12) Patent Application Publication
(10) Pub. No.: US 2025/0352085 A1
(43) Pub. Date: Nov. 20, 2025

(54) DETECTING APPARATUS, DETECTING METHOD AND NON-TRANSITORY RECORDING MEDIUM

(71) Applicant: NEC Corporation, Minato-ku, Tokyo (JP)

(72) Inventors: Shizuo SAKAMOTO, Tokyo (JP); Akita TSUCHIYA, Tokyo (JP)

(73) Assignee: NEC Corporation, Minato-ku, Tokyo (JP)

(21) Appl. No.: 18/833,444

(22) PCT Filed: Feb. 9, 2022
(86) PCT No.: PCT/JP2022/005093

§ 371 Date: Jan. 22, 2025

(51) Int. Cl. A61B 5/107; A61B 5/00

(52) U.S. Cl. CPC A61B 5/107; A61B 5/441; A61B 5/742

(57) ABSTRACT

A detecting apparatus 1 comprises a acquiring part 11 acquiring three dimensions information 3DI indicating a three-dimensional shape of skin of a finger, and a detecting part 12 detecting a damaged part of the finger on the basis of the three dimensions information 3DI.

This application is a National Stage Entry of PCT/JP2022/005093 filed on Feb. 9, 2022, the contents of all of which are incorporated herein by reference, in their entirety.

### TECHNICAL FIELD

This disclosure relates to technical fields of a detecting apparatus, a detecting method and a recording medium.

### BACKGROUND ART

It is described in a patent literature 1 that a technique acquires image information indicating an image of a nipple layer, and studying a peculiar area indicating damage of the nipple layer. It is described in a patent literature 2 that a technique acquires peculiar area information indicating a peculiar area included in a living body pattern, and displays the peculiar area with a display attribute, which differs from the living body pattern other than the peculiar area on the basis of the peculiar area information. It is described in a patent literature 3 that a technique acquires information of a peculiar area, which is detected on the basis of living body pattern information, of the living body pattern information, controls such that the living body information is displayed by giving display attributes, that are different from each other, an area being the peculiar area and an area other than the peculiar area on the basis of information about the peculiar area. It is described in a patent literature 4 that a technique measures a three-dimensional position of a finger surface on the basis of data of the finger surface including a fingerprint, obtains an axis direction of a finger tip on the basis of the measured three-dimensional position, sets a curve coordinate system forming a curved surface formed from a first crossing line group of a vertical cross section group, which is approximately parallel to a finger tip axis direction, and the finger surface, and a second crossing line group of a lateral cross section group, which is approximately perpendicular to the vertical cross section group, and the finger surface, obtains intermediate data expressed in the curve coordinate system from a fingerprint image expressed in a predetermined plane coordinate system, and obtains matching data expressed in a coordinate system of a virtual plane obtained by virtually expanding a curved surface corresponding to the curve coordinate system from the intermediate data.

### CITATION LIST

### Patent Literature

Patent Literature 1: International Publication No. 2016/204176
Patent Literature 2: International Publication No. 2016/159390
Patent Literature 3: Japanese Patent Application Laid Open No. 2020-080188
Patent Literature 4: Japanese Patent Application Laid Open No. 2006-172258

### SUMMARY

### Technical Problem

This disclosure is to provide a detecting apparatus, a detecting method and a recording medium aiming to improve techniques described in the prior art literatures.

### Solution to Problem

One aspect of a detecting apparatus comprises: an acquiring means for acquiring three dimensions information indicating a three-dimensional shape of skin of a finger, and a detecting means for detecting a damaged part of the finger on the basis of the three dimensions information.

One aspect of a detecting method includes: acquiring three dimensions information indicating a three-dimensional shape of skin of a finger, and detecting a damaged part of the finger on the basis of the three dimensions information.

One aspect of a recording medium records a computer program, wherein the computer program makes a computer perform a detecting method including acquiring three dimensions information indicating a three-dimensional shape of skin of a finger, and detecting a damaged part of the finger on the basis of the three dimensions information.

### BRIEF DESCRIPTION OF THE DRAWINGS

FIG. 1 is a block diagram showing a configuration of a detecting apparatus in a first embodiment.

FIG. 2 is a block diagram showing a configuration of a detecting apparatus in a second embodiment.

FIG. 3A is a conceptual scheme of a fingerprint.

FIG. 3B is a conceptual scheme of a fingerprint.

FIG. 3C is a conceptual scheme of a fingerprint.

FIG. 3D is a conceptual scheme of a fingerprint.

FIG. 4 is a flowchart showing flow of detecting operation performed by the detecting apparatus in the second embodiment.

FIG. 5A is a figure showing an example of a first characteristic damaged part.

FIG. 5B is a figure showing an example of a first characteristic damaged part.

FIG. 6A is a figure showing an example of a second characteristic damaged part.

FIG. 6B is a figure showing an example of a second characteristic damaged part.

FIG. 7A is a figure showing an example of a third characteristic damaged part.

FIG. 7B is a figure showing an example of a third characteristic damaged part.

FIG. 8 is a block diagram showing a configuration of a detecting apparatus in a fourth embodiment.

FIG. 9 is a block diagram showing a configuration of a detecting apparatus in a fifth embodiment.

FIG. 10 is a block diagram showing a configuration of a detecting apparatus in a sixth embodiment.

FIG. 11A is a displaying example by the detecting apparatus of the sixth embodiment.

FIG. 11B is a displaying example by the detecting apparatus of the sixth embodiment.

FIG. 12 is a block diagram showing a configuration of a detecting apparatus in a seventh embodiment.

FIG. 13 is a conceptual scheme of restricting operation in the seventh embodiment.

### DESCRIPTION OF EXAMPLE EMBODIMENTS

Embodiments of a detecting apparatus, a detecting method and a recording medium are described hereinafter with referring figures.

### 1: First Embodiment

A first embodiment of a detecting apparatus, a detecting method and a recording medium is described. Hereinafter, the first embodiment of the detecting apparatus, the detecting method and the recording medium is described using a detecting apparatus 1 to which the first embodiment of the detecting apparatus, the detecting method and the recording medium is applied.

### [1-1: Configuration of the Detecting Apparatus 1]

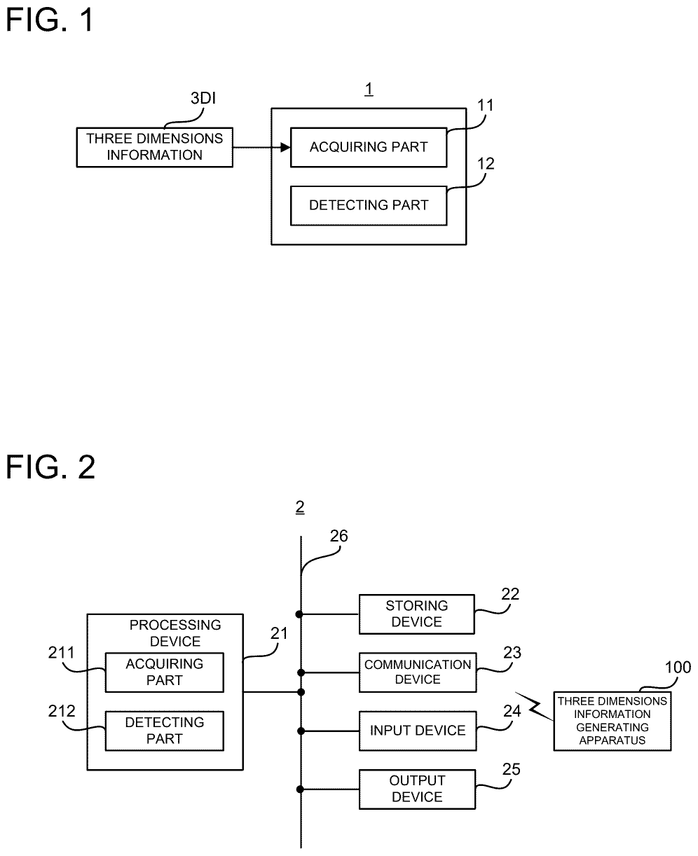

A configuration of the detecting apparatus 1 in the first embodiment is described with referring to FIG. 1. FIG. 1 is a block diagram showing the configuration of the detecting apparatus 1 in the first embodiment.

As shown in FIG. 1, the detecting apparatus 1 comprises an acquiring part 11 and a detecting part 12. The acquiring part 11 acquires three dimensions data 3DI indicating a three-dimensional shape of a skin of a finger. The detecting unit 12 detects a damaged part of the skin on the basis of the three dimensions information 3DI.

### [1-2: Technical Effect of the Detecting Apparatus 1]

Since a damaged part of a skin is detected on the basis of the three dimensions information 3DI indicating a three-dimensional shape of the skin of a finger, it is possible to accurately detect the damaged part of the skin compared to when the three-dimensional shape of the skin of the finger is not used.

### 2: Second Embodiment

A second embodiment of a detecting apparatus, a detecting method and a recording medium is described. Hereinafter, the second embodiment of the detecting apparatus, the detecting method and the recording medium is described using a detecting apparatus 2 to which the second embodiment of the detecting apparatus, the detecting method and the recording medium is applied.

### [2-1: Configuration of the Detecting Apparatus 2]

A configuration of the detecting apparatus 2 in the second embodiment is described with referring to FIG. 2. FIG. 2 is a block diagram showing the configuration of the detecting apparatus 2 in the second embodiment.

As shown in FIG. 2, the detecting apparatus 2 comprises a processing device 21 and a storing device 22. The detecting apparatus 2 may further comprise a communication device 23, an input device 24 and an output device 25. However, the detecting apparatus 2 may not comprise at least one of the communication device 23, the input device 24 and the output device 25. The processing device 21, the storing device 22, the communication device 23, the input device 24 and the output device 25 may be connected through a data bus 26.

The processing device 21 includes at least one of a CPU (Central Processing Unit), a GPU (Graphics Processing Unit) and an FPGA (Field Programmable Gate Array), for example. The processing device 21 reads computer programs. For example, the processing device 21 may read a computer program stored in the storing device 22. For example, the processing device 21 may read a computer program recorded on a computer readable and non-transitory recording medium by using a not shown recording medium reading device (e.g., the input device 24 described later) comprised in the detecting apparatus 2. The processing device 21 may acquire (in other words, download or read) a computer program from a not shown apparatus outside of the detecting apparatus 2 through the communication device 23 (or another communication device). The processing device 21 executes read computer programs. As a result, logical functional blocks for performing operation to be performed by the detecting apparatus 2 are realized in the processing device 21. In other words, the processing device 21 can function as a controller to realizing logical functional blocks for performing operation (i.e., process) to be performed by the detecting apparatus 2.

FIG. 2 shows one example of logical functional blocks realized in the processing device 21 for performing detecting operation. As shown in FIG. 2, in the processing device 21, an acquiring part 211, which is one specific example of an “acquiring means”, and a detecting part 212, which is one specific example of a “detecting means”, are realized. Operation of each of the acquiring part 211 and the detecting part 212 is described later.

The storing device 22 can store desired data. For example, the storing device 22 may temporally store computer programs executed by the processing device 21. The storing device 22 may temporally store data temporally used by the processing device 21 when the processing device 21 executes a computer program. The storing device 22 may store data to be stored for a long time by the detecting apparatus 2. Wherein, the storing device 22 may include at least one of a RAM (Random Access Memory), a ROM (Read Only Memory), a hard disk device, an optical magnetic disk device, an SSD (Solid State Drive), and a disk array device. In other words, the storing device 21 may include non-transitory recording medium.

The communication device 23 can communicate with an apparatus outside of the detecting apparatus 2 through a not shown communication network. The communication device 23 may acquire three dimensions information 3DI that is used in detecting operation from a three dimensions information generating apparatus 100 described later through the communication network.

The input device 24 is a device receiving information input to the detecting apparatus 2 from outside of the detecting apparatus 2. For example, the input device 24 may include an operation device (e.g., at least one of a keyboard, a mouse and a touch panel) which can be operated by an operator of the detecting apparatus 2. For example, the input device 24 may include a reading device, which can read information recorded, as data, on a recording medium being able to externally attach to the detecting apparatus 2.

The output device 25 is a device outputting information to outside of the detecting apparatus 2. The output device 25 may output information as images. In other words, the output device 25 may include a display device (so-called display) which can display an image indicating outputted information. For example, the output device 25 may output information as sound. In other words, the output device 25 may include a sound device (so-called speaker) which can output sound. For example, the output device 25 may output information to papers. In other words, the output device 25 may include a printing device (so-called printer) which can print desired information to papers.

### [2-2: Operated Finger]

An illegal action for forging a fingerprint which is a pattern of a skin of a finger is performed. A typical example of the illegal action is an action in which a person, who is cited in a blacklist, forges a fingerprint and passes through an examination such as in an immigration examination.

There is a method for surgically changing a fingerprint, as a method for forging. For example, a method for creating chemical burn by burning a part of or all of a fingerprint with chemical agents, a method for transplanting a fingerprint of another finger such as transplanting a fingerprint of a finger of a foot to a fingerprint of a finger of a hand, and a method for changing a fingerprint by damaging a skin such as a method for shifting a position relationship of a fingerprint such as pulling a skin by making a rift on a finger area known. A finger, of which a fingerprint is surgically changed as described above, is also called an “operated finger”.

There is a method in which an operated finger is detected by detecting a state such as discontinuity of ridge lines from plane fingerprint images. It is considered that it is determined a damaged finger having damage when a pattern included in a fingerprint image is not a pattern by ridge lines and valley lines. However, when a plane fingerprint image is observed, there may be a case in which it be seen like a damaged finger having damage even if it is a normal finger not having damage. For example, there may be a case in which discontinuity of ridge lines is detected even if it is a normal finger not having damage due to alteration by such as overworking fingers of a hand, and naturally changing such as forming age-related wrinkles. For example, when a state of discontinuity of ridge lines is detected from a plane fingerprint image, there may be a case in which it is difficult to distinguish between discontinuity due to naturally changing and unnatural discontinuity due to such as damage intendedly created by such as an operation.

By the way, a fingerprint is formed by a shape of derma. Therefore, when an epiderm is damaged, a fingerprint is reproduced. Thus, it is needed to damage derma for forging a fingerprint by intendedly damaging skin. In other words, it is considered that an operated finger, which is created by intendedly damaging skin, is damaged down to at least derma.

It is known that deeply damaged skin turns into a scar having hard connective tissue by increasing collagen fibers over passage of time, and then, cicatricial contracture is caused by contracting over passage of time. A hard, swollen and contracted damaged part of skin is also called “pulling around skin”. Cicatricial contracture occurs regardless of size of a scar. Therefore, it is possible to determine whether or not there is wound history when cicatricial contracture can be detected, even if a careful operation has been performed.

As described above, since the operated finger has been damaged down to derma, cicatricial contracture has occurred in the operated finger. Therefore, it is considered that there is likely that a finger, in which cicatricial contracture is found, is an operated finger. In other words, it is suitable for detecting an operated finger that if cicatricial contracture of skin of a finger is found at taking fingerprint.

When skin has cicatricial contracture, a state of undulating of a skin surface differs compered to another part without cicatricial contracture. For example, there is often a case, in which a bump or a dent of a part having cicatricial contracture is extremely greater than that of another part. It also can be said that there is often a case, in which a part having cicatricial contracture has lost regularity of unevenness of a ridge pattern in another part.

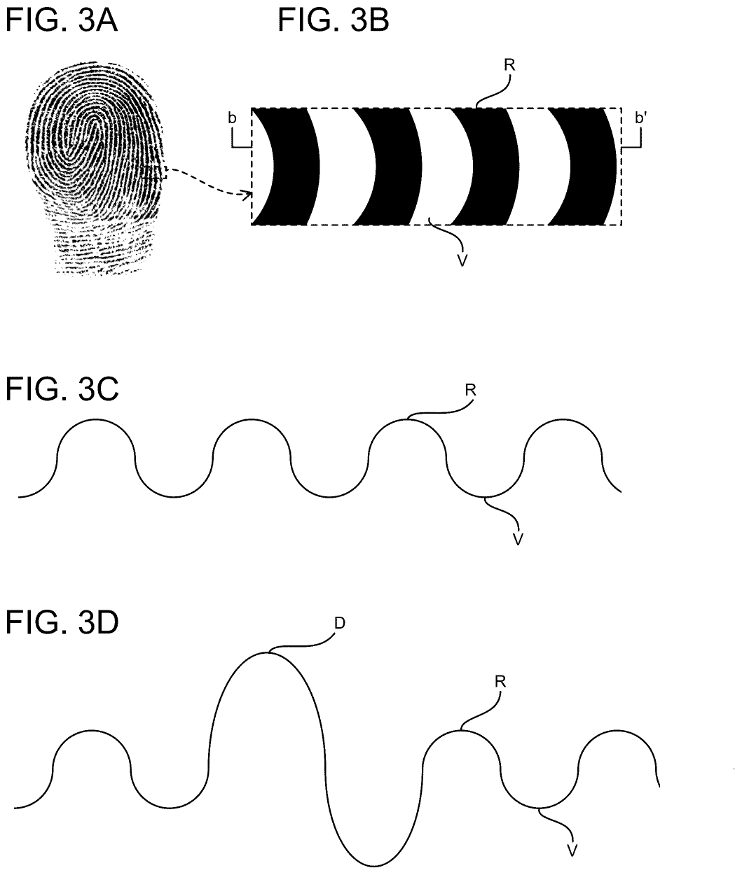

FIG. 3A to FIG. 3D is a conceptual scheme of a fingerprint. FIG. 3A is a conceptual scheme of a two-dimensional pattern of skin of a finger. FIG. 3A is a top view of a fingerprint when a skin surface is seen from above, and does not include information about a dimension in a normal direction of a finger surface.

FIG. 3B is a figure showing a part of the skin surface shown in FIG. 3A. FIG. 3C is a cross-sectional view taken along a line b-b′ in FIG. 3B, and is a conceptual scheme of a three-dimensional pattern of a skin including the dimension in the normal direction of the finger surface. FIG. 3C shows undulating of the fingerprint.

FIG. 3B and FIG. 3C exemplify a normal finger without damage. In FIG. 3B and FIG. 3C, curves indicate ridge lines R, and gaps between curves indicate valley lines V. As shown in FIG. 3B, when it is the normal finger, width of each of ridge lines R is almost the same, and each gap between ridge lines R is almost the same, and a ridge line direction of each of ridge lines R continues. Moreover, as shown in FIG. 3C, when it is the normal finger, undulations with the same height difference continue at approximately equal intervals.

On the other hand, FIG. 3D is a conceptual scheme of a three-dimensional pattern, which includes a dimension in a normal direction of a finger surface, of skin of a damaged finger having damage. It exemplifies that height difference in a damaged part D is large compared to an adjacent part.

Moreover, since a part having cicatricial contracture has a different property from normal skin, it may be determined that there is a damaged part when there is unevenness, which differs from ridge lines. In this way, a damaged part may be detected by detecting information about a depth direction of skin. Moreover, since there is often a case, in which a pitch in a part having cicatricial contracture is extremely narrower or wider than another part, it may be determined that there is a damaged part by detecting such a part.

### [2-3: Detecting Operation Performed by the Detecting Apparatus 2]

In consideration of the above-mentioned characteristics of the operated finger, the detecting apparatus 2 in the second embodiment measures a three-dimensional shape of skin of a finger, and detects the operated finger on the basis of a measurement result.

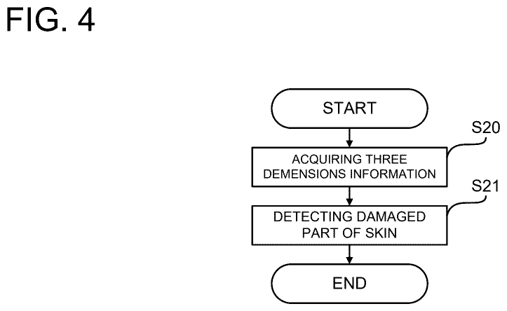

Flow of detecting operation performed by the detecting apparatus 2 in the second embodiment is described with referring to FIG. 4. FIG. 4 is a flowchart showing the flow of the detecting operation performed by the detecting apparatus 2 in the second embodiment.

The acquiring part 211 acquires three dimensions information 3DI indicating a three-dimensional shape of skin of a finger (step S20). A height difference of undulations of skin of a finger of a general adult man is less than or equal to 50 mm. Thus, the acquiring part 211 may acquire the three dimensions information 3DI indicating a three-dimensional shape about from a surface of skin of a finger to approximately 50 mm depth.

The acquiring part 211 may acquire the three dimensions information 3DI from a three dimensions information generating apparatus 100 through at least one of the communication device 23 and the input device 24. Such as an optical coherence tomography (OCT) imaging apparatus, and an infrared camera may be used as the three dimensions information generating apparatus 100. The three dimensions information generating apparatus 100 may generate the three dimensions information 3DI indicating a three-dimensional shape of skin of a part of a finger. The three dimensions information generating apparatus 100 may generate the three dimensions information 3DI indicating a three-dimensional shape of skin of a pad of a finger. Moreover, the three dimensions information generating apparatus 100 may generate the three dimensions information 3DI indicating a three-dimensional shape of skin of a part of a finger. Moreover, the three dimensions information generating apparatus 100 may generate the three dimensions information 3DI indicating a three-dimensional shape of all skin of a finger.

The detecting part 212 detects a damaged part of skin on the basis of the three dimensions information 3DI (step S21). The detecting part 212 may determine that a part, undulating degree of skin (called “roughness”) of which greatly differs from another part, is the damaged part. When there is a part, roughness of which differs by more than or equal to a predetermined amount from roughness of another part, the detecting part 212 may detect the part as the damaged part on the basis of the three dimensions information 3DI.

Roughness may be a parameter including a dimension of a height direction. The height direction may be a normal direction of a skin surface. Roughness may include at least one of an elevate difference of undulations and thickness of skin.

The elevate difference of undulations may be a difference between a top of a ridge and a bottom of a valley of skin. The ridge may include a ridge line, and the valley may include a valley line. The ridge line may indicate a part, in which an opening of a sweat gland on skin raises. The valley line may be a groove between adjacent ridge lines.

Roughness may include depth of a valley of skin. In this case, the detecting part 212 may detect a part, which has a deeper or shallower valley by a predetermined amount than a valley of an adjacent part, as the damaged part.

Roughness may include height of a top of a ridge of skin. The detecting part 212 may detect a part, which has a lower or higher ridge by a predetermined amount than a ridge of an adjacent part, as the damaged part.

It is possible to obtain a cross-sectional image of skin including epiderm and derma by OCT imaging, and to calculate thickness of skin, thereby roughness may include the thickness of the skin. The detecting part 212 may detect a part, thickness of skin of which is thicker or thinner by a predetermined amount than thickness of an adjacent part, as the damaged part.

### [2-4: Technical Effect of the Detecting Apparatus 2]

According to the detecting apparatus 2 in the second embodiment, since roughness of skin is measured, it is possible to accurately detect a damaged part of skin. Moreover, by using a height difference of undulations as the roughness, it is possible to detect an unnatural damaged part by surgical history.

### 3: Third Embodiment

A third embodiment of a detecting apparatus, a detecting method and a recording medium is described. Hereinafter, the third embodiment of the detecting apparatus, the detecting method and the recording medium using a detecting apparatus 3 to which the third embodiment of the detecting apparatus, the detecting method and the recording medium is applied.

The detecting apparatus 3 in the third embodiment differs detecting operation of the detecting part 212 compared to the detecting apparatus 2 in the second embodiment. Other features of the detecting apparatus 3 may be the same as other features of the detecting apparatus 2.

In the third embodiment, the detecting part 212 detects a damaged part on the basis of a three dimensions information 3DI even when there are no parts, in which height degree of undulations of skin differs by a predetermined amount from height degree of undulations of an adjacent part. In other words, in the third embodiment, the detecting part 212 may detect abnormalities in two-dimensional space. The detecting part 212 may detect the damaged part of skin by combining information of a normal direction of a skin surface and information of two-dimensional space of the skin surface. The detecting part 212 may detect a damaged part of skin on the basis of a state of a two-dimensional fingerprint formed by ridge lines. The detecting part 212 may detect whether or not there is a damaged part on the basis of a state of unevenness of skin, which is smaller than a state of unevenness of so-called “cicatricial contracture”.

As described above, FIG. 3B and FIG. 3C are figures exemplify a normal figure without a damage part. Therefore, when there is a part, which does not have features, exemplified in FIG. 3B and FIG. 3C, the detecting part 212 may detect the part as a damaged part. When at least one of a first characteristic damaged part, a second characteristic damaged part and a third characteristic damaged part, as described bellow, is detected, the detecting part 212 may detect the part as a damaged part.

### [3-1: Example of the First Characteristic Damaged Part]

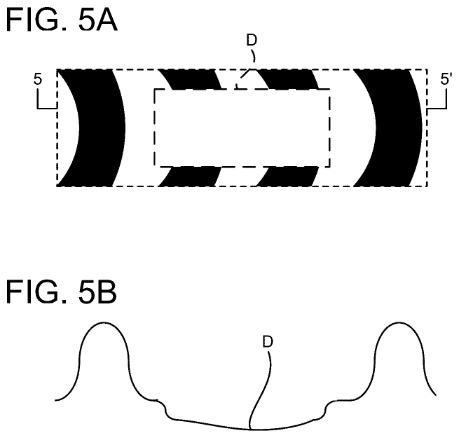

When there is a part, in which a distance between adjacent ridge lines on skin differs by a predetermined amount from a distance between adjacent ridge lines of an adjacent part, the detecting part 212 may detect the part on the basis of the three dimensions information 3DI. FIG. 5A and FIG. 5B is a figure exemplifies the first characteristic damaged part, in which a distance between ridge lines is abnormal. FIG. 5A is a top view of a fingerprint when a part of a skin surface is seen from above. FIG. 5B is a cross-sectional view taken along a line 5-5′ in FIG. 5A showing undulations of the fingerprint. As shown in FIG. 5A, when there is a part D, in which a distance between ridge lines R is abnormal compared to the case shown in FIG. 3B, the detecting part 212 may detect the part as a damaged part D. As shown in FIG. 5B, when there is a part D, in which a distance between ridge lines R is extremely wide, it may be determined that a height difference of undulations caused by cicatricial contracture is reduced by scraping off the cicatricial contracture, which is a surgical scar. Alternatively, when there is a part, in which undulating degree of skin is lower than a valley V in an adjacent part, the detecting part 212 may detect the part as a damaged part D formed by scraping off cicatricial contracture, which is a surgical scar. As shown in FIG. 5B, when there is a part, in which a distance between ridge lines R is abnormal, there may be a part, in which undulations are flat, and the detecting part 212 may detect the part as a damaged part D.

Moreover, when skin is lost, unevenness of the skin is also lost. The detecting part 212 may detect a damaged part D on the basis of disappearance of unevenness. The detecting part 212 can detect an operated finger even when there has been damage that results in loss of skin.

### [3-2: Example of the Second Characteristic Damaged Part]

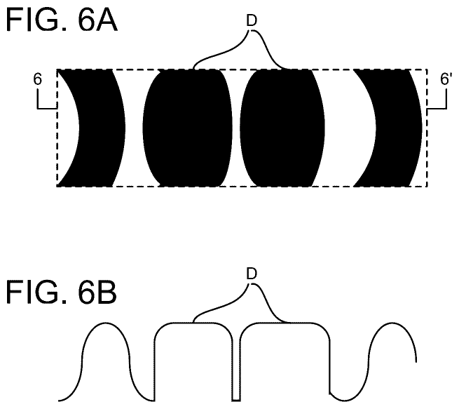

When there is a part, in which width of a ridge line on skin differs by a predetermined amount from width of a ridge line of an adjacent part, the detecting part 212 may detect the part as a damaged part on the basis of the three dimensions information 3DI. FIG. 6A and FIG. 6B is a figure showing an example of the second characteristic damaged part, in which width of a ridge line is abnormal. FIG. 6A is a top view of a fingerprint when a skin surface is seen from above. FIG. 6B is a cross-sectional view taken along a line 6-6′ in FIG. 6A showing undulations of the fingerprint. As shown in FIG. 6A, when there is a part D, in which width of a ridge line R is abnormal compared to the case shown in FIG. 3B, the detecting part 212 may detect the part as a damaged part D. As shown in FIG. 6B, when there is a part, in which width of the ridge line R is extremely wide, it may be determined that a height difference of undulations caused by cicatricial contracture is reduced by pulling the cicatricial contracture, which is a surgical scar. As shown in FIG. 6B, when there is the part, in which the width of the ridge line R is abnormal, undulations having the same height difference may exist at intervals different from those of an adjacent part. In other words, when there is a part, in which ridge line pitches are non-uniform, the detecting part 212 may detect the part as a damaged part D.

### [3-3: Example of the Third Characteristic Damaged Part]

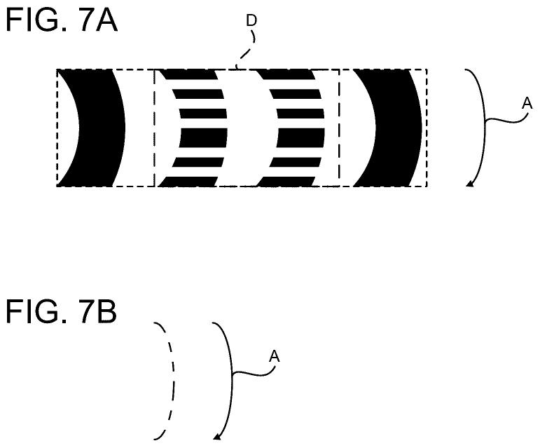

When there is a portion, in which there are parts each of which has a continuous ridge line on skin, length of which is less than or equal to a first threshold value, wherein a number of the parts is more than or equal to a second threshold value, the detecting part 212 may detect the portion as a damaged part on the basis of the three dimensions information 3DI. FIG. 7A and FIG. 7B is a figure showing an example of the third characteristic damaged part, in which continuity of ridge lines is abnormal. FIG. 7A is a top view of a fingerprint when a skin surface is seen from above. FIG. 7B is a figure showing continuity of a direction of an arrow A of a ridge line shown in FIG. 7A. As shown in FIG. 7A, when a part D, in which continuity of a ridge line R is abnormal compared to the case shown in FIG. 3B, the detecting part 212 may detect the part as a damaged part. When it is determined that there is a part having discontinuity in a ridge direction A shown in FIG. 7B, the detecting part 212 may detect the part as a damaged part.

### [3-4: Technical Effect of the Detecting Apparatus 3]

It is possible to detect a damaged part even when, for example, a surgical scar is further concealed by such as scraping off cicatricial contracture, by detecting abnormality of a distance between adjacent ridge lines on skin when there is no abnormality in a height difference of undulations.

It is possible to detect a damaged part even when, for example, a surgical scar is further concealed by such as pulling cicatricial contracture, by detecting width of a ridge line on skin when there is no abnormality in a height difference of undulations.

Discontinuous ridge lines are often detected in damaged parts of skin. Therefore, it is possible to more accurately detect a damaged part of skin by detecting discontinuous ridge lines.

### 4: Fourth Embodiment

A fourth embodiment of a detecting apparatus, a detecting method and a recording medium is described. Hereinafter, the fourth embodiment of the detecting apparatus, the detecting method and the recording medium is described using a detecting apparatus 4 to which the fourth embodiment of the detecting apparatus, the detecting method and the recording medium is applied.

### [4-1: Configuration of the Detecting Apparatus 4]

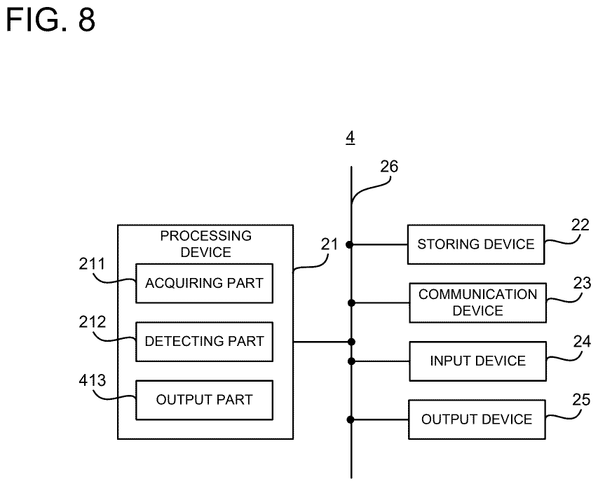

A configuration of the detecting apparatus 4 is described with referring to FIG. 8. FIG. 8 is a block diagram showing the configuration of the detecting apparatus 4 in the fourth embodiment.

As shown in FIG. 8, the detecting apparatus 4 in the fourth embodiment differs that the processing device 21 comprises an output part 413 compared to the detecting apparatus 2 in the second embodiment and the detecting apparatus 3 in the third embodiment. Other features of the detecting apparatus 4 may be the same as other features of the detecting apparatus 2 and the detecting apparatus 3.

The output part 413 outputs a detection result of the detecting part 212. The output part 413 may output the detection result of the detecting part 212 through the output device 25. The output part 413 may output the detection result of the detecting part 212 by controlling at least any of a display, a speaker and a printer as the output device 25.

### [4-2: Technical Effect of the Detecting Apparatus 4]

An administrator of the detecting apparatus 4 can quickly recognize presence of an authenticated person with a damaged finger by outputting the detection result of the detecting part 212.

### 5: Fifth Embodiment

A fifth embodiment of a detecting apparatus, a detecting method and a recording medium is described. Hereinafter, the fifth embodiment of the detecting apparatus, the detecting method and the recording medium is described using a detecting apparatus 5 to which the fifth embodiment of the detecting apparatus, the detecting method and the recording medium is applied.

### [5-1: Configuration of the Detecting Apparatus 5]

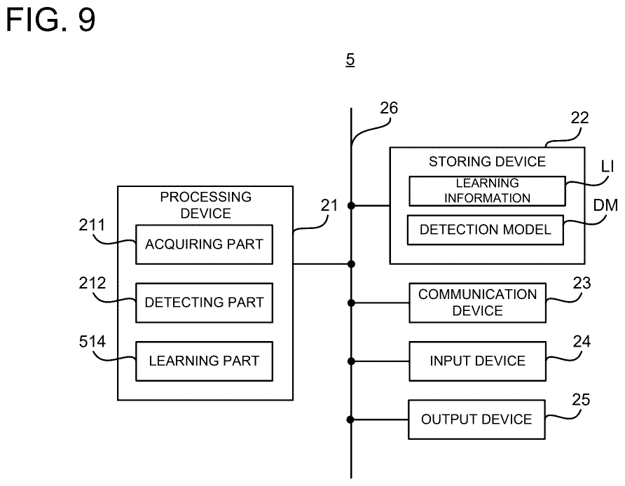

A configuration of the detecting apparatus 5 in the fifth embodiment is described with referring to FIG. 9. FIG. 9 is a block diagram showing the configuration of the detecting apparatus 5 in the fifth embodiment.

As shown in FIG. 9, the detecting apparatus 5 in the fifth embodiment differs that the processing device 21 comprises a learning part 514 and the storing device 22 stores learning information LI compared to the detecting apparatus 2 in the second embodiment—the detecting apparatus 4 in the fourth embodiment. However, the storing device 22 may not store the learning information LI. The learning information LI is described later. Other features of the detecting apparatus 5 may be the same as other features of the detecting apparatus 2—the detecting apparatus 4.

There is a high demand for high accurately detecting whether or not there is a damaged part which is a surgical scar. Therefore, in the fifth embodiment, a detection model DM, which can high accurately detect a damaged part, is constructed by machine learning. The detecting part 212 may detect a damaged part by using the detection model DM.

### [5-2: Learning Operation Performed the Detecting Apparatus 5]

The acquiring part 211 acquires the learning information LI. The learning information LI includes sample shape information indicating a three-dimensional shape of a finger of a sample person, and a correct label indicating whether or not skin of the finger of the sample person is damaged. The detecting part 212 detects a damaged part of the skin of the sample person.

The learning part 514 makes the detection model DM learn a detecting method for a damaged part on the basis of the correct label and the detection result of a damaged part of the skin of the sample person by the detecting part 212. The learning part 514 may make the detection model DM learn the detecting method for the damaged part such that a difference (an error) between the correct label and the detection result of the damaged part of the skin of the sample person by the detecting part 212 becomes small or minimized. The learning part 514 may make the detection model DM learn the detecting method for the damaged part such that the correct label matches the detection result of the damaged part of the skin of the sample person by the detecting part 212. For example, the learning part 514 may make the detection model DM used by the detecting part 212 learn the detecting method for the damaged part such that the detection model DM detects the damaged part of the skin of the sample person when the correct label indicates that there is a damaged part. Alternatively, the learning part 514 may make the detection model DM used by the detecting part 212 learn the detecting method for the damaged part such that the detection model DM does not detect that the skin of the sample person has a damaged part when the correct label indicates that there are no damaged parts.

When the three dimensions information 3DI indicating a three-dimensional shape of skin of a finger is inputted in the detection model DM, the detection model DM may output information indicating whether or not there is a damaged part of skin of the finger. The learning information LI may include sample shape information indicating a three-dimensional shape of each of parts of a finger of the sample person, and correct label indicating whether or not skin of each of parts of the finger of the sample person is damaged. The detection model DM may learn whether or not there is a damaged part depending on undulation degree of skin of a finger.

Parameters defining operation of the detection model DM may be stored in the storing device 22. The parameters defining the operation of the detection model DM may be parameters updated by learning operation, and may be weights and biases of a neural network, for example.

The learning information LT may not indicate a three-dimensional shape of a finger including a damaged part. The detection model DM may be learned such that (1) when the three dimensions information 3DI indicating a three-dimensional shape of a finger having a damaged part is inputted, a detection result closes to a result “a damaged area present” indicated by a correct label, and when the three dimensions information 3DI indicating a three-dimensional shape of a finger without a damaged part is inputted, a detection result closes to a result “no damaged part” indicated by a correct label.

### [5-3: Technical Effect of the Detecting Apparatus 5]

The detecting part 212 can higher accurately detect a damaged part by performed machine learning.

### 6: Sixth Embodiment

A sixth embodiment of a detecting apparatus, a detecting method and a recording medium is described. Hereinafter, the sixth embodiment of the detecting apparatus, the detecting method and the recording medium is described using a detecting apparatus 6 to which the sixth embodiment of the detecting apparatus, the detecting method and the recording medium is applied. [6-1: Configuration of the detecting apparatus 6]

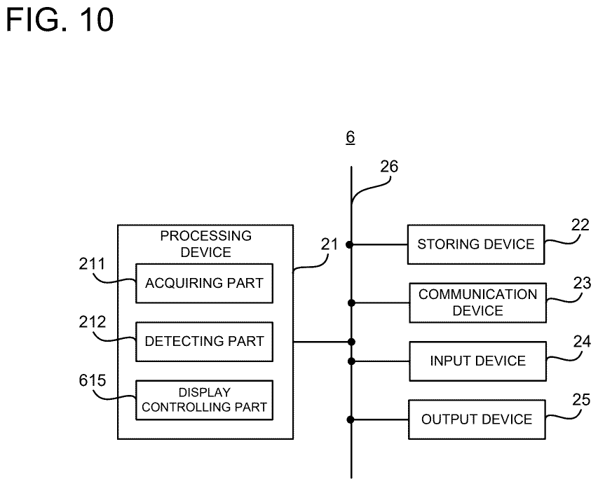

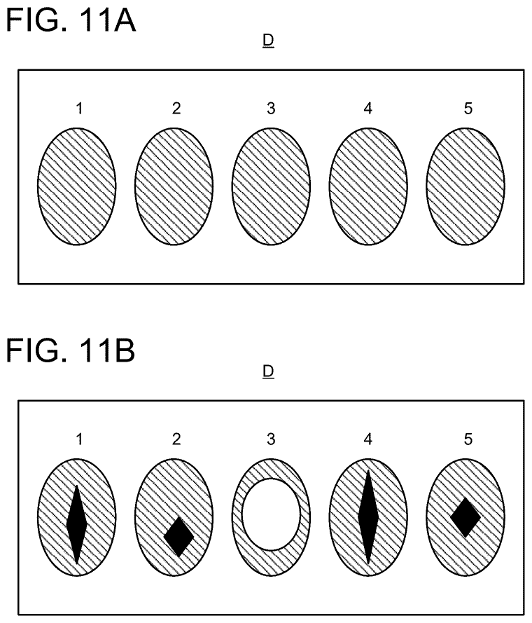

A configuration of the detecting apparatus 6 in the sixth embodiment is described with referring to FIG. 10. FIG. 10 is a block diagram showing the configuration of the detecting apparatus 6 in the sixth embodiment.

As shown in FIG. 10, the detecting apparatus 6 in the sixth embodiment differs that the processing device 21 comprises a display controlling part 615 compared to the detecting apparatus 2 in the second embodiment—the detecting apparatus 5 in the fifth embodiment. Other features of the detecting apparatus 6 may be the same as other features of the detecting apparatus 2—the detecting apparatus 5.

In the sixth embodiment, the three dimensions information generating apparatus 100 may generate the three dimensions information 3DI indicating three-dimensional shapes of skin of a first finger-a fifth finger of one hand.

### [6-2: Example of Display Operation Performed by the Detecting Apparatus 6]

The display controlling part 615 makes a display D display an image showing roughness of skin on the basis of the three dimensions information 3DI.

FIG. 11A and FIG. 11B shows an example of an image showing roughness of skin of a finger displayed on an administrator's display D. As shown in FIG. 11A and FIG. 11B, the image showing the roughness of the skin may be an image, in which degree of shading changes according to undulating degree. FIG. 11A and FIG. 11B show roughness of skin of tips of a first finger-a fifth finger of one hand. FIG. 11A shows a displaying example of the display D when hands of an authenticated person do not have an operated finger. Since undulations of all fingers are average, all images of 1-5 have the same density. FIG. 11B shows a displaying example of the display D when hands of an authenticated person have operated fingers. Undulations of a first, a second, a fourth and a fifth fingers are partially too raised, so they are displayed partially as black. Moreover, undulations of a third finger are partially too recessed, so they are displayed partially as white.

The display controlling part 615 may display, as the image showing roughness of skin, an image in which a height difference from a bottom of a valley to a top of a ridge is represented by changes in color, wherein the valley and the ridge are adjacent to each other. The display controlling part 615 may convert roughness of skin into a heat map on the basis of the three dimensions information 3DI, and may display it on a screen of the administrator's display D. When there is a part, undulating degree of skin of which differs by a predetermined amount from undulating degree in an adjacent part, the display controlling part 615 may display an image in which the part blinks.

### [6-3: Technical Effect of the Detecting Apparatus 6]

An administrator can easily determine whether or not it is an abnormal finger having a surgical scar by visually checking the administrator's display D.

### 7: Seventh Embodiment

A seventh embodiment of a detecting apparatus, a detecting method and a recording medium is described. Hereinafter, the seventh embodiment of the detecting apparatus, the detecting method and the recording medium is described using a detecting apparatus 7 to which the seventh embodiment of the detecting apparatus, the detecting method and the recording medium is applied.

### [7-1: Configuration of the Detecting Apparatus 7]

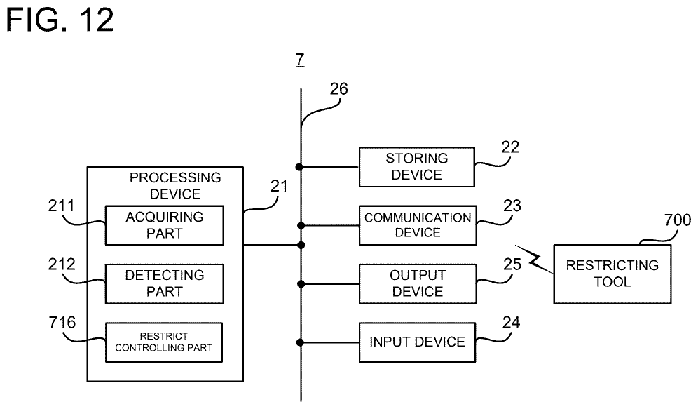

A configuration of the detecting apparatus 7 in the seventh embodiment is described with referring to FIG. 12. FIG. 12 is a block diagram showing the configuration of the detecting apparatus 7 in the seventh embodiment.

As shown in FIG. 12, the detecting apparatus 7 in the seventh embodiment differs that the processing device 21 comprises a restrict controlling part 716 compared to the detecting apparatus 2 in the second embodiment—the detecting apparatus 6 in the sixth embodiment. Other features of the detecting apparatus 7 may be the same as other features of the detecting apparatus 2—the detecting apparatus 6.

### [7-2: Example of Restricting Operation Performed by the Detecting Apparatus 7]

In the seventh embodiment, a restricting tool 700 may be displaced near a location where a wrist contacts when a fingerprint is taken. In the seventh embodiment, a loading table T, on which at least a hand, fingers and a wrist are placed, may be provided in order to image the hand and fingers using the three dimensions information generating apparatus 100. The restricting tool 700 may be displaced near a location on the loading table T where the wrist contacts.

The restricting tool 700 may be a tool temporarily restricting motion of an authenticated person in a certain case. The restrict controlling part 716 may control movement of the restricting tool 700 through at least one of the communication device 23 and the output device 25.

In the seventh embodiment, the three dimensions information generating apparatus 100 may generate the three dimensions information 3DI indicating three-dimensional shapes of skin of a first finger-a fifth finger of one hand. The restrict controlling part 716 may control the restricting tool 700 to restrict motion of an authenticated person when the detecting part 212 detects a damaged part of skin from a predetermined number of fingers of each finger of a hand. The restricting tool 700 may restrict a hand of an authenticated person.

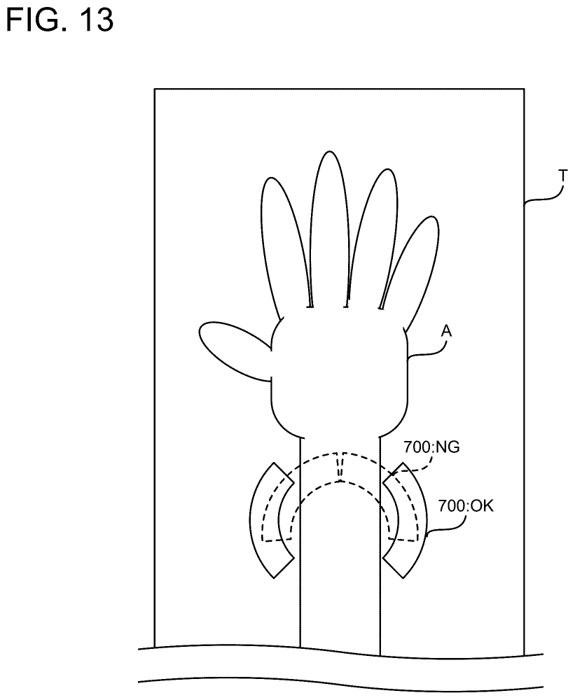

As shown in FIG. 13, the restrict controlling part 716 may control operation of the restricting tool 700 to bring a state in which a wrist is not restricted as shown by solid lines (700: OK) when the detecting part 212 determines that a hand of an authenticated person is a hand without an operated finger. On the other hand, the restrict controlling part 716 may control the operation of the restricting tool 700 to bring a state in which a wrist is restricted as shown by dashed lines (700: NG) when the detecting part 212 determines that a hand of an authenticated person is a hand having an operated finger. The restrict controlling part 716 may operate the restricting tool 700 when operated finger are detected in a predetermined number of fingers such as four or more.

### [7-3: Technical Effect of the Detecting Apparatus 7]

For example, when an illegal entrant is discovered at an airport, it is possible to prevent that the illegal entrant escapes.

### 8: Supplementary Note

In regard to embodiments described above, the following supplementary notes may be further disclosed.

### [Supplementary Note 1]

A detecting apparatus comprising:
an acquiring means for acquiring three dimensions information indicating a three-dimensional shape of skin of a finger; and
a detecting means for detecting a damaged part of the finger on the basis of the three dimensions information.

### [Supplementary Note 2]

The detecting apparatus according to the supplementary note 1, wherein
when there is a part, in which undulating degree of the skin differs by more than or equal to a predetermined amount compared with undulating degree of an adjacent part, on the basis of the three dimensions information, the detecting means detects the part as the damaged part.

### [Supplementary Note 3]

The detecting apparatus according to the supplementary note 2, wherein
the undulating degree includes at least one of a height difference of undulating and thickness of the skin.

### [Supplementary Note 4]

The detecting apparatus according to any one of supplementary notes 1 to 3, wherein
When there is a part, in which a distance between adjacent ridge lines on the skin differs by more than or equal to a predetermined amount compared with a distance between adjacent ridge lines of an adjacent part, on the basis of the three dimensions information, the detecting means detects the part as the damaged part.

### [Supplementary Note 5]

The detecting apparatus according to any one of supplementary notes 1 to 4, wherein
when there is a part, in which width of a ridge line on the skin differs by more than or equal to a predetermined amount compared with width of a ridge line of an adjacent part, on the basis of the three dimensions information, the detecting means detects the part as the damaged part.

### [Supplementary Note 6]

The detecting apparatus according to any one of supplementary notes 1 to 5, wherein
when there is a portion, in which there are parts each of which has a continuous ridge line on the skin, length of which is less than or equal to a first threshold value, wherein a number of the parts is more than or equal to a second threshold value, on the basis of the three dimensions information, the detecting means detects the portion as the damaged part.

### [Supplementary Note 7]

The detecting apparatus according to any one of supplementary notes 1 to 6, wherein
the detecting apparatus further comprise an output means for outputting a detection result of the detecting means.

### [Supplementary Note 8]

The detecting apparatus according to any one of supplementary notes 1 to 7, wherein
the acquiring means acquires learning information including sample shape information indicating a three-dimensional shape of a finger of a sample person and a correct label indicating whether or not skin of the finger of the sample person is damaged,
the detecting means detects a damaged part of the skin of the sample person on the basis of the sample shape information, and
the detecting apparatus further comprises a learning means for making the detecting means learn a detecting method for the damaged part on the basis of the correct label and the detection result of the damaged part of the skin of the sample person by the detecting means.

### [Supplementary Note 9]

The detecting apparatus according to any one of supplementary notes 1 to 8, further comprising:
a displaying means for displaying an image showing undulating degree of the skin on the basis of the three dimensions information.

### [Supplementary Note 10]

The detecting apparatus according to any one of supplementary notes 1 to 9, wherein
the acquiring means acquires three dimensions information indicating a three-dimensional shape of skin of each of fingers of a hand, and
the detecting apparatus further comprises a restricting means for restricting motion of an authenticated person having the hand when the detecting means detects a damaged part of the skin from predetermined number of fingers from each of fingers of the hand.

### [Supplementary Note 11]

A detecting method including:
acquiring three dimensions information indicating a three-dimensional shape of skin of a finger; and
detecting a damaged part of the finger on the basis of the three dimensions information.

### [Supplementary Note 12]

A recording medium in which a computer program is recorded, wherein the computer program makes a computer perform a detecting method including acquiring three dimensions information indicating a three-dimensional shape of skin of a finger, and detecting a damaged part of the finger on the basis of the three dimensions information.

At least a part of components of each of the above-mentioned embodiments may be combined with at least another part of components of each of the above-mentioned embodiments. A part of components of each of the above-mentioned embodiments may be not used. In addition, to the extent permitted by law, the disclosures of all documents (e.g., publications) cited in this disclosure above are incorporated by reference into this disclosure.

This disclosure can appropriately be changed within limits being not contrary to summary of inventions or ideas, that can be read from the scope of claims and all of the specification, a detecting apparatus, a detecting method and a recording medium with such changes are also included in technical ideas of this disclosure.

### DESCRIPTION OF REFERENCE CODES

1, 2, 3, 4, 5, 6, 7 Detecting apparatus
11, 211 Acquiring part
12, 212 Detecting part
413 Output part
514 Learning part
615 Display controlling part
716 Restrict controlling part
100 Three dimensions information generating apparatus
LI Learning information
DM Detection model
D Display

What is claimed is:

1. A detecting apparatus comprising:
at least one memory configured to store instructions, and
at least one processor configured to execute the instructions to:
acquire three dimensions information indicating a three-dimensional shape of skin of a finger; and
detect a damaged part of the finger on the basis of the three dimensions information.

2. The detecting apparatus according to claim 1, wherein
when there is a part, in which undulating degree of the skin differs by more than or equal to a predetermined amount compared with undulating degree of an adjacent part, on the basis of the three dimensions information, the at least one processor is configured to execute the instructions to detect the part as the damaged part.

3. The detecting apparatus according to claim 2, wherein
the undulating degree includes at least one of a height difference of undulating and thickness of the skin.

4. The detecting apparatus according to claim 1, wherein
when there is a part, in which a distance between adjacent ridge lines on the skin differs by more than or equal to a predetermined amount compared with a distance between adjacent ridge lines of an adjacent part, on the basis of the three dimensions information, the at least one processor is configured to execute the instructions to detect the part as the damaged part.

5. The detecting apparatus according to claim 1, wherein
when there is a part, in which width of a ridge line on the skin differs by more than or equal to a predetermined amount compared with width of a ridge line of an adjacent part, on the basis of the three dimensions information, the at least one processor is configured to execute the instructions to detect the part as the damaged part.

6. The detecting apparatus according to claim 1, wherein
when there is a portion, in which there are parts each of which has a continuous ridge line on the skin, length of which is less than or equal to a first threshold value, wherein a number of the parts is more than or equal to a second threshold value, on the basis of the three dimensions information, the at least one processor is configured to execute the instructions to detect the portion as the damaged part.

7. The detecting apparatus according to claim 1, wherein
the at least one processor is configured to execute the instructions to:
acquire learning information including sample shape information indicating a three-dimensional shape of a finger of a sample person and a correct label indicating whether or not skin of the finger of the sample person is damaged,
a damaged part of the skin of the sample person on the basis of the sample shape information, and
learn a detecting method for the damaged part on the basis of the correct label and the detection result of the damaged part of the skin of the sample person by the detecting means.

8. The detecting apparatus according to claim 1,
wherein the at least one processor is configured to execute the instructions to display an image showing undulating degree of the skin on the basis of the three dimensions information.

9. The detecting apparatus according to claim 1, wherein
the at least one processor is configured to execute the instructions to:
acquire three dimensions information indicating a three-dimensional shape of skin of each of fingers of a hand, and
restrict motion of an authenticated person having the hand when a damaged part of the skin is detected from a predetermined number of fingers from each of fingers of the hand.

10. A detecting method including:
acquiring three dimensions information indicating a three-dimensional shape of skin of a finger; and
detecting a damaged part of the finger on the basis of the three dimensions information.

11. A non-transitory recording medium in which a computer program is recorded, wherein the computer program makes a computer perform a detecting method including acquiring three dimensions information indicating a three-dimensional shape of skin of a finger, and detecting a damaged part of the finger on the basis of the three dimensions information.

12. The detecting apparatus according to claim 2, wherein
when there is a part, in which a distance between adjacent ridge lines on the skin differs by more than or equal to a predetermined amount compared with a distance between adjacent ridge lines of an adjacent part, on the basis of the three dimensions information, the at least one processor is configured to execute the instructions to detect the part as the damaged part.

13. The detecting apparatus according to claim 3, wherein
when there is a part, in which a distance between adjacent ridge lines on the skin differs by more than or equal to a predetermined amount compared with a distance between adjacent ridge lines of an adjacent part, on the basis of the three dimensions information, the at least one processor is configured to execute the instructions to detect the part as the damaged part.

14. The detecting apparatus according to claim 2, wherein
when there is a part, in which width of a ridge line on the skin differs by more than or equal to a predetermined amount compared with width of a ridge line of an adjacent part, on the basis of the three dimensions information, the at least one processor is configured to execute the instructions to detect the part as the damaged part.

15. The detecting apparatus according to claim 3, wherein
when there is a part, in which width of a ridge line on the skin differs by more than or equal to a predetermined amount compared with width of a ridge line of an adjacent part, on the basis of the three dimensions information, the at least one processor is configured to execute the instructions to detect the part as the damaged part.

16. The detecting apparatus according to claim 4, wherein
when there is a part, in which width of a ridge line on the skin differs by more than or equal to a predetermined amount compared with width of a ridge line of an adjacent part, on the basis of the three dimensions information, the at least one processor is configured to execute the instructions to detect the part as the damaged part.

17. The detecting apparatus according to claim 12, wherein
when there is a part, in which width of a ridge line on the skin differs by more than or equal to a predetermined amount compared with width of a ridge line of an adjacent part, on the basis of the three dimensions information, the at least one processor is configured to execute the instructions to detect the part as the damaged part.

18. The detecting apparatus according to claim 13, wherein
when there is a part, in which width of a ridge line on the skin differs by more than or equal to a predetermined amount compared with width of a ridge line of an adjacent part, on the basis of the three dimensions information, the at least one processor is configured to execute the instructions to detect the part as the damaged part.

19. The detecting apparatus according to claim 2, wherein
when there is a portion, in which there are parts each of which has a continuous ridge line on the skin, length of which is less than or equal to a first threshold value, wherein a number of the parts is more than or equal to a second threshold value, on the basis of the three dimensions information, the at least one processor is configured to execute the instructions to detect the portion as the damaged part.

20. The detecting apparatus according to claim 3, wherein
when there is a portion, in which there are parts each of which has a continuous ridge line on the skin, length of which is less than or equal to a first threshold value, wherein a number of the parts is more than or equal to a second threshold value, on the basis of the three dimensions information, the at least one processor is configured to execute the instructions to detect the portion as the damaged part.
